(19) United States
(12) Patent Application Publication
(10) Pub. No.: US 2005/0084528 A1
(43) Pub. Date: Apr. 21, 2005

(54) Anti-platelet aggregation compositions

(76) Inventors: Sheikh Arshad Saeed, Woodford Green (GB); Shahzada Omar Saeed, Woodford Greeen (GB)

Correspondence Address: BACON & THOMAS, PLLC, 625 SLATERS LANE, FOURTH FLOOR, ALEXANDRIA, VA, 22314, US

(21) Appl. No.: 10/718,665

(22) Filed: Nov. 24, 2003

(30) Foreign Application Priority Data
Oct. 15, 2003 (GB) 0324213.8

(51) Int. Cl. A61K 31/60; A61K 9/20; A61K 31/18

(52) U.S. Cl. 424/464; 514/165; 514/602

(57) ABSTRACT

A combination of nimesulide and a sub-therapeutic dose of aspirin provides effective anti-platelet aggregation treatment.

This invention relates to pharmaceutical compositions for application as anti-platelet aggregation agents. In particular, the invention is concerned with the use of compositions comprising aspirin and nimesulide wherein the aspirin is present at a sub-therapeutic dose.

Platelet aggregation is involved in a number of cardiovascular, disorders (e.g. myocardial infarction and angina) which are a major cause of ill health among the human population. Accordingly a number of different anti-platelet aggregation agents have been investigated for use in the treatment and/or prevention of such diseases.

Platelet aggregation is, however, a poorly understood and complex phenomenon which appears to be associated with a plethora of different metabolic pathways. This is reflected in the fact that a whole range of agonists [e.g. collagen, platelet activating factor (PAF), arachadonic acid (AA), thrombin, adenosine diphosphate (ADP), calcium ionophore (A23187) and epinephrine (epi)] have been shown to induce platelet aggregation. One challenge to be overcome in developing effective anti-platelet aggregation agents is providing a means to effect inhibition of the pathway which has been activated by the agonist present.

One drug which is widely used in anti-platelet aggregation therapy is aspirin (CAS No. 50-78-2). It is sometimes used, for example, in the treatment of unstable angina and myocardial infarction. Aspirin is also believed to be of value in preventing or reducing the risk of second or further adverse cardiovascular events and is commonly prescribed in such a situation for daily intake.

The use of aspirin in combination with a COX II inhibitor for anti-platelet aggregation has also been disclosed in U.S. Pat. No. 6,511,968, where compositions comprising a therapeutically effective amount of aspirin (here given as 75-325 mg) and a therapeutically effective amount of a COX II inhibitor are described for use in treating, preventing or reducing the risk of a number of conditions such as acute coronary ischemic syndrome, thrombosis, thromboembolism, thrombotic occlusion, restenosis, transient ischemic attack and stroke. The rationale behind inclusion of a COX II inhibitor in the anti-platelet aggregation agent is not entirely clear, but the suggestion seems to be that its anti-inflammatory effect is beneficial in reducing the occurrence of adverse cardiovascular events. Thus a huge number of COX II inhibitors of quite disparate structure are mentioned in the patent, though none are exemplified and only the combinations of aspirin with 3-phenyl-4-(4-(methylsulfonyl)phenyl)-2-(5H)-furanone and with 5-chloro-3-(4-(methylsulfonyl)phenyl)-2-(2-methyl-5-pyridyl)pyridine are specifically defined in the claims.

One of the COX II inhibitors mentioned in passing in U.S. Pat. No. 6,511,968 is nimesulide (CAS No. 51803-78-2). Nimesulide is a non-steroidal anti-inflammatory drug (NSAID) which is most commonly used in the treatment of inflammatory conditions, fever and/or pain. It has also been reported that nimesulide can function as an anti-platelet aggregation agent (Saeed et al., Biochemical Society Transactions 1998, 26, S342 and Life Sciences 1998, 63(20), pp. 1835-1841). These studies show that nimesulide, and likewise aspirin, inhibit platelet aggregation induced by AA and PAF, and that nimesulide also inhibits platelet aggregation induced by adrenaline.

However, the use of aspirin, whether alone or in combination with a COX II inhibitor, does have potential major drawbacks. The use of therapeutic doses of aspirin (typically 75 mg or more) is commonly associated with gastro-intestinal disturbances (e.g. nausea, dyspepsia, vomiting) and can also cause gastric mucosal damage such as ulceration. Dizziness, tinnitus, deafness and sweating are also known to occur with larger and/or repeated doses. In extreme cases of aspirin overdose, cardiovascular collapse and respiratory failure may result.

A further problem associated with the use of aspirin in anti-platelet aggregation therapy is that whilst it is effective in inhibiting platelet aggregation induced by AA and PAF, it may be less effective against certain other agonists. Accordingly there is a need for alternative anti-platelet aggregation agents which can provide a broader spectrum of activity (i.e. activity against a wider range of agonists) and which are associated with fewer side effects.

It has now surprisingly been found that the use of aspirin in combination with nimesulide enhances the level of suppression of platelet aggregation relative to the use of either of the agents alone, even when aspirin is used at a sub-therapeutic dose. In particular, an unexpected synergistic effect may be observed between nimesulide and a sub-therapeutic dose of aspirin. This synergistic effect, even at sub-therapeutic doses of aspirin, has significant clinical implications since the use of less aspirin reduces or avoids the side effects associated with this drug, without compromising the therapeutic efficacy achieved. Thus the therapeutic efficacy (i.e. the anti-platelet aggregation effect) of this combination of agents may be enhanced relative to use of either of the agents alone. More particularly, the therapeutic efficacy may be synergistically enhanced.

Thus viewed from one aspect the invention provides a pharmaceutical composition comprising nimesulide and a sub-therapeutic dose of aspirin, optionally together with one or more pharmaceutically acceptable carriers, excipients and/or diluents.

Viewed from a further aspect the invention provides the use of nimesulide and a sub-therapeutic dose of aspirin in the manufacture of a medicament for application as an anti-platelet aggregation agent.

Alternatively viewed the invention provides a method of treatment of a human or non-human animal subject in need of anti-platelet aggregation treatment, said method comprising administering to said subject an effective amount of a pharmaceutical composition comprising nimesulide and a sub-therapeutic dose of aspirin.

Nimesulide and aspirin for use according to the invention may be obtained from commercial sources, e.g. from Sigma-Aldrich or Cayman Chemicals Co. USA, or may be prepared using standard processes and procedures well known to those skilled in the art. The compounds have the formulae:

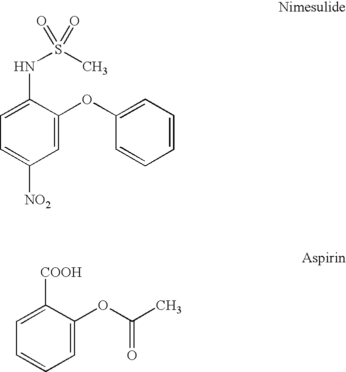

Nimesulide is also known as 4′-nitro-2′-phenoxymethanesulphonanilide and N-(4-nitro-2-phenoxyphenyl)methanesulfonamide and the term nimesulide is used herein synonymously with both of these terms.

Aspirin is also known as acetylsalicylic acid, acidum acetylsalicylicum, salicylic acid acetate, o-acetylsalicylic acid and 2-acetoxybenzoic acid. The term aspirin is used herein synonymously with each of the aforementioned terms.

Side effects of nimesulide are rare and it has generally been found to be well tolerated in a wide range of patients, including elderly and young patients prone to showing side effects with other NSAIDs. Nimesulide has also been tried as a labour-delaying drug and it has been speculated that it may produce fewer adverse side effects on the foetus than other NSAIDs. Use of nimesulide also has the advantage that it is less acidic than many other NSAIDs (pKa 6.5) and has a rapid onset of action.

In preferred compositions of the invention the nimesulide and aspirin may be formulated in a conventional manner with one or more inert carriers, excipients and/or diluents. Examples of carriers, excipients and diluents include water, ethanol, glycerol, polyethylene glycol, sodium chloride, sugars (e.g. glucose, sucrose or lactose), starches (e.g. corn starch or maize starch), microcrystalline cellulose, gums (e.g. gum tragacanth), sorbitol, mannitol, xylitol, magnesium stearate, polyvinylpyrrolidone, fatty acids (e.g. stearic acid), fats, waxes, calcium carbonate, calcium chloride and citric acid.

The compositions may if desired additionally comprise one or more wetting agents, sweetening agents (e.g. a sugar, aspartame or saccharin), lubricating agents, emulsifying agents, suspending agents, preserving agents, flavouring agents (e.g. vanillin, peppermint oil or a fruit flavouring) and/or absorption enhancers.

Preferred compositions of the invention may additionally comprise further (e.g. one or two) pharmaceutically active substances. An advantage associated with the use of a combination of aspirin, nimesulide and further (e.g. one) active substance(s) is that it may increase the spectrum of diseases for which the composition is suitable for use as a medicament. Additionally or alternatively, an even further lowering of the dose of aspirin and/or the further active substance may advantageously be achieved. This is particularly advantageous if the dose of aspirin which is associated with known above-described side effects can be further reduced.

Additional active substances which may be used in the compositions of the invention include NSAIDs, e.g. COX I and/or COX II inhibitors. Representative COX I and II inhibitors include ibuprofen, mefenamic acid, indomethacin, naproxen, diclofenac salts, paracetamol, N-acetyl-2-aminophenol, N-acetyl-3-aminophenol, rofecoxib, meloxicam, celecoxib, piroxicam, nabumetone, ketoprofen, flusulide, NS-398 and etodolac. In one preferred embodiment the additional active substance may be indomethacin and/or naproxen. Generally, indomethacin may be used in amounts of 1-50 mg per dosage form, in particular about 5-25 mg per dosage form, whilst naproxen may be used in amounts of 10-500 mg per dosage form, for example about 50-250 mg per dosage form.

Other active substances which may be present in the compositions of the invention include vitamins (e.g. vitamin E and in particular vitamin C) and/or CNS stimulants (e.g. caffeine).

The quantity of aspirin present in the compositions of the invention is sub-therapeutic. “Sub-therapeutic” may be considered to be an amount of aspirin which, if administered on its own, would not suffice to achieve a beneficial therapeutic effect (e.g. anti-platelet aggregation). Such an amount may be less than 75 mg, in particular less than 60 mg, e.g. less than 30 mg, per dosage form.

Generally, aspirin may be used in amounts of from 1-60 mg per dosage form, e.g. from 1-50 or 2-50 mg per dosage form, such as about 5-40 mg per dosage form, in particular about 8-30 mg per dosage form (e.g. about 10 or 15 mg per dosage form). A dosage form may be considered to be one quantum of medicine (e.g. per tablet or per measured amount of liquid). Aspirin may be conveniently used in amounts of from 5-40 wt % of the composition, preferably 10-30 wt %.

The quantity of nimesulide present in the compositions of the invention may be readily determined by those skilled in the art and will depend on several factors, including the method of administration and the weight of the subject. Generally, however, nimesulide may be used in amounts of from 1-200 mg per dosage form, more preferably 5-100 mg per dosage form, in particular about 10-50 mg per dosage form (e.g. 12-25 mg per dosage form, such as about 12 mg per dosage form). In some preferred compositions, the amount of nimesulide present is sub-therapeutic (e.g. less than 100 mg per dosage form, preferably less than 75 mg per dosage form, e.g. about 25 or 12.5 mg per dosage form). Nimesulide may be conveniently used in amounts of from 20-80 wt % of the composition, preferably 40-60 wt %.

In particularly preferred compositions the amount of nimesulide and aspirin present is that required to provide the combination with a therapeutic efficacy which is enhanced relative to use of either of the agents alone. In other words, it is preferred that the combination of nimesulide and aspirin according to the present invention exhibits at least a complementary or additive therapeutic efficacy (i.e. anti-aggregation effect).

Especially preferred combinations of nimesulide and aspirin are those which demonstrate synergistically enhanced therapeutic efficacy. Synergism may result in a therapeutic efficacy which is greater than the additive effect achieved by summation of the use of each of aspirin and nimesulide alone. Synergistic compositions are particularly advantageous since they allow for the lowering of the dose of aspirin and/or nimesulide (especially of aspirin), and therefore allow for a reduction in the extent and/or frequency of side effects to be achieved.

Representative examples of compositions exhibiting a synergistically enhanced therapeutic efficacy comprise 1-60 mg aspirin and 1-200 mg nimesulide, preferably 1-50 mg aspirin and 5-100 mg nimesulide, more preferably 8-30 mg aspirin and 10-50 mg nimesulide, e.g. 10-15 mg aspirin and 12-25 mg nimesulide, typically 10 mg aspirin and 12.5 mg nimesulide.

It will also be appreciated that the aspirin and the nimesulide need not be provided in the same composition in order for their synergistic effect to be achieved in vivo. Thus the sub-therapeutic dose of aspirin and the dose of nimesulide may alternatively be provided in separate preparations which are administered either simultaneously or sequentially.

Thus viewed from a further aspect the invention provides a preparation comprising nimesulide and, separately, a sub-therapeutic dose of aspirin, for simultaneous or sequential use as an anti-platelet aggregation agent. In preferred preparations, the doses of nimesulide and aspirin present are as hereinbefore described in relation to pharamaceutical compositions of the invention.

The compositions and preparations according to the invention may be administered orally, rectally (e.g. using a suppository), topically or systemically. The route chosen will depend, for example, on the subject to be treated. Preferably the compositions and preparations according to the invention are administered orally.

The compositions and preparations of the invention may be presented in any form adapted for use in the administration route selected. Forms suitable for systemic administration may, for example, be formulations for intradermal, intraperitoneal or intravenous injection or infusion.

Forms adapted for topical administration include compositions for administration to the skin and mucosa (e.g. gels, creams, sprays, lotions, salves and aerosols).

Forms suitable for oral administration include, for example, plain or coated tablets, sustained release tablets, chewable tablets, soft capsules, hard capsules, suspensions and syrups. Preferred forms for use according to the invention are tablets, suspensions or syrups, particularly tablets.

Compositions and preparations of the present invention are preferably in a ready to use form. Alternatively the composition or preparations may be provided in a concentrate form which is mixed with a liquid (e.g. hot water) immediately prior to use.

Compositions and preparations of the invention may also be in forms adapted to permit slow release of nimesulide. This may, for example, be achieved through encapsulation of the nimesulide content in biodegradable polymer microparticles or microsponge delivery systems.

The compositions hereinbefore described are suitable for the therapeutic or prophylactic treatment of cardiovascular disorders. A cardiovascular disorder may be considered to be a condition of the heart, arteries or veins which disrupts the supply of oxygen to life-sustaining areas of the body such as the brain, the heart, etc. Examples of cardiovascular disorders include angina pectorisis, stroke, myocardial infarction, arteriosclerosis, arrhythmia and thrombosis (including thrombosis induced by surgery). Preferably, the compositions of the invention are used for the prophylactic treatment of cardiovascular disorders (e.g. myocardial infarction).

All preferred features of the compositions hereinbefore described are also preferred features of medicaments produced using of nimesulide and aspirin in accordance with the present invention. Thus the invention also provides use of nimesulide and a sub-therapeutic dose of aspirin in the manufacture of a medicament for treatment of any of the above-mentioned diseases.

Furthermore, all preferred features of the preparations hereinbefore described are also preferred features of preparations manufactured using nimesulide and aspirin in accordance with the invention. Consequently use of nimesulide and, separately, a sub-therapeutic dose of aspirin in the manufacture of a preparation as hereinbefore defined for simultaneous or sequential use as an anti-platelet aggregation agent forms a further aspect of the invention.

The invention will now be described in more detail by way of the following non-limiting Example:

### EXAMPLE

Anti-Platelet Aggregation Action of Aspirin and Nimesulide

In a randomized observer-blinded crossover study with a 2 week washout period, young age-matched male volunteers (n=34) took a single dose of (i) aspirin (10, 20 or 40 mg), (ii) nimesulide (5, 12.5, 25 or 50 mg) or (iii) aspirin and nimesulide (10 mg and 12.5 mg respectively), with a 250 ml glass of water, on an empty stomach. 10 ml blood samples were taken, by vein puncture, just prior to, and 24 hours after, drug ingestion, and the samples were mixed with 3.8% w/v sodium citrate solution (9:1) and centrifuged at 260 g for 15 minutes at 20° C. to obtain platelet rich plasma (PRP).

The PRP samples were treated in vitro with calcium ionophore (A23187, 2.5 μM) or arachidonic acid (AA, 1.6 mM) to induce maximal aggregation. Platelet aggregation was monitored using a Dual-channel Lumi-aggregometer (Model 400, Chronolog Corporation, USA) using 0.45 ml samples of PRP. The resulting aggregation was recorded for all of the samples and a comparison was made between the pre- and post-test drug ingestion aggregation levels. The data was analysed using the Student's t-test and differences were considered significant when probability (p) was <0.05.

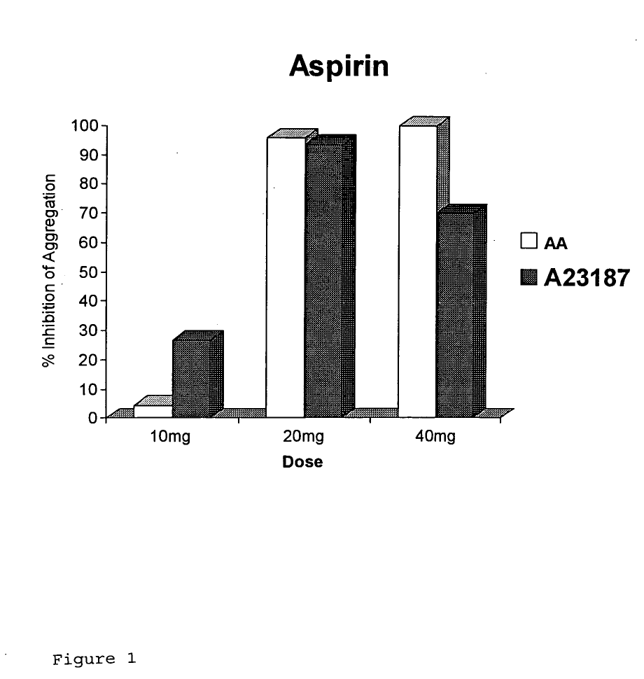

The results of the ex vivo study show that both aspirin and nimesulide inhibit platelet aggregation induced by AA or A23187 in a concentration dependent manner. At a dose of 20 mg or 40 mg, aspirin effectively inhibited platelet aggregation induced by either agonist, whereas at a concentration of 10 mg, aspirin was only weakly effective (about 25% inhibition) against platelet aggregation induced by A23187 and was ineffective against AA (see FIG. 1).

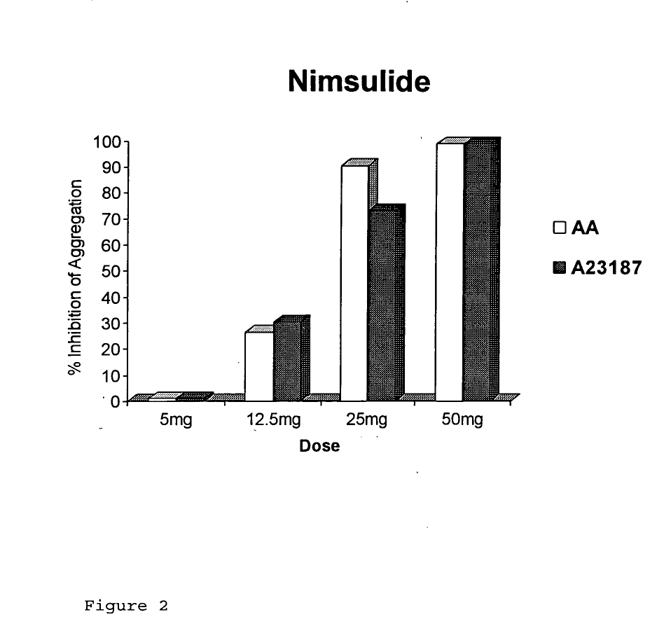

Nimesulide, at doses of 25 or 50 mg, also effectively inhibited platelet aggregation induced by AA and A23187, but only effected approximately 30% inhibition against each agonist at a dose of 12.5 mg (see FIG. 2).

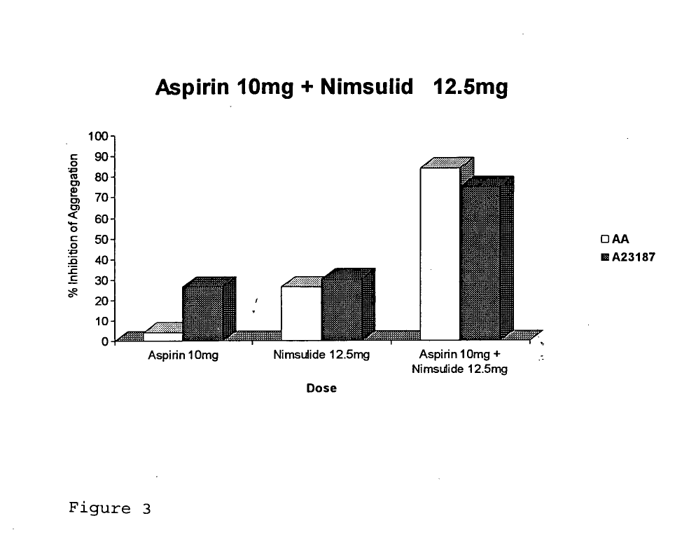

The results obtained from use of aspirin and nimesulide in combination are startling (see FIG. 3). The use of 10 mg of aspirin and 12.5 mg of nimesulide in combination was found to inhibit platelet aggregation induced by AA and A23187 at levels of approximately 85% and 75% respectively. Thus, use of nimesulide and aspirin as a combination leads to synergistically enhanced therapeutic efficacy relative to the use of either compound alone. This is further illustrated by Table 1 below.

TABLE 1
Anti-Platelet Aggregation Effect of Aspirin, Nimesulide
and a combination thereof In Vivo

| | % Inhibition | % Inhibition |
|---|---|---|
| | AA | A23187 |
| Aspirin 10 mg | 4 | 27 |
| Nimesulide 12.5 mg | 28 | 32 |
| Additive effect (of | 32 | 59 |
| aspirin and nimesulide) | | |
| Actual effect (of aspirin | 85 | 75 |
| and nimesulide) | | |

1. A pharmaceutical preparation comprising nimesulide and a sub-therapeutic dose of aspirin.

2. The preparation of claim 1 wherein said nimesulide and said aspirin are formulated separately for simultaneous or sequential administration.

3. The preparation of claim 1 wherein said nimesulide and said aspirin are formulated together as a single composition.

4. The preparation of claim 1 wherein said nimesulide and said aspirin are present in amounts such that the preparation has an anti-platelet aggregation effect exceeding that of either nimesulide or aspirin alone.

5. The preparation of claim 4 wherein the amounts of said nimesulide and said aspirin are such that their anti-platelet aggregation effect is synergistic.

6. The preparation of claim 1 wherein said sub-therapeutic dose of aspirin is 1-60 mg per dosage form.

7. The preparation of claim 1 wherein said nimesulide is present in an amount of 1-200 mg per dosage form.

8. The preparation of claim 1 wherein said nimesulide is present in a sub-therapeutic amount per dosage form.

9. A method of treatment of a human or non-human animal subject in need of anti-platelet aggregation treatment, said method comprising administering to said subject an effective amount of a pharmaceutical preparation comprising nimesulide and a sub-therapeutic dose of aspirin.
